(19) United States
(12) Patent Application Publication
(10) Pub. No.: US 2010/0189785 A1
(43) Pub. Date: Jul. 29, 2010

(54) Use of D-ribose for fatigued subjects

(75) Inventors: John A. St. Cyr, Coon Rapids, MN (US); Dean J. MacCarter, Englewood, CO (US)

Correspondence Address: MUETING, RAASCH & GEBHARDT, P.A., P.O. BOX 581336, MINNEAPOLIS, MN, 55458-1336, US

(73) Assignee: Bioenergy, Inc., Ham Lake, MN (US)

(21) Appl. No.: 12/583,430

(22) Filed: Aug. 20, 2009

Related U.S. Application Data
(60) Provisional application No. 61/208,122, filed on Feb. 20, 2009.
(60) Provisional application No. 61/189,498, filed on Aug. 20, 2008.

(51) Int. Cl. A61K 9/22; A61K 31/70; A61P 9/00; A61P 11/00; A61P 3/00

(52) U.S. Cl. 424/468; 514/23

(57) ABSTRACT

Low doses of D-ribose raise the level of fitness and lower the perception of fatigue in baby boomers aged 45-65 who perceive themselves as fatigued. The doses range from 0.100 grams to 3.0 grams, bid, for a total of 0.200 to 6.0 grams daily. Objective measures of cardiopulmonary parameters confirm the improvement of fitness and questionnaires confirm that quality of life and mental states are improved with D-ribose administration.

### RELATED APPLICATIONS

This application claims priority of U.S. Provisional Applications Ser. Nos. 61/189,498, filed Aug. 20, 2008 and 61/208,122, filed Feb. 20, 2009.

### BACKGROUND OF THE INVENTION

Over the last twenty years, many studies have shown the benefit of oral or intravenous administration of D-ribose, a naturally occurring pentose carbohydrate, to restore energy levels in persons in whom ATP levels are low due to cardiac ischemia, congestive heart failure, poor pulmonary function and other such conditions. Healthy persons undergoing increased demand for energy such as those exercising intensely, have also benefited from D-ribose supplementation.

The energy coinage of the cell is adenosine triphosphate (ATP). During anabolism, the energy derived from the metabolism of nutrients is transferred to high energy phosphate bonds of ATP. The energy in these bonds is expended during the energy consumption phase. During energy consumption, ATP loses one high energy bond to form ADP, which can be hydrolyzed to AMP. AMP and its metabolites adenine, hypoxanthine, xanthine and inosine are freely diffusible from the muscle cell and may not be available for resynthesis to ATP via the salvage pathway. The energy buildup steps occur within the cell during two basic processes. Oxidative phosphorylation replenishes ATP by the breakdown and phosphorylation of circulating fatty acids, glucose and intramuscular glycogen and triglycerides, through the Krebs tricarboxylic acid cycle, with oxygen as a terminal electron acceptor. Anaerobic phosphorylation provides ATP via the Emden-Meyerhoff pathway of glycolysis derived from circulating glucose and intramuscular glycogen via kinase reactions such as the myokinase reaction. Lactic acid is the final product of anaerobic glycolysis.

Regardless of whether the high energy phosphate bonds of ATP are generated oxidatively or anaerobically, and irrespective of the substrates used for its generation, ATP cannot be synthesized unless the precursors of the ATP molecule itself are available. The resynthesis of the ATP molecule can occur by either de novo or salvage pathways. Synthesis by the de novo pathway is slow. Ribose is found in the normal diet only in very low amounts, and is synthesized from glucose within the body by the pentose phosphate pathway. In the de novo synthetic pathway, ribose is phosphorylated to 5-phosphoribosyl-1-phosphate pyrophosphate (PRPP), and condensed with adenine to form the intermediate adenosine monophosphate (AMP). AMP is further phosphorylated via high energy bonds to form adenosine diphosphate (ADP) and ATP. Normally, AMP synthesis is believed to occur mainly by the salvage pathway, however, following anoxia or ischemia where breakdown products diffuse from the cells, the activity of the de novo pathway is increased.

In the synthesis of ATP via the nucleotide salvage pathway, the nucleotide precursors that may be present in the tissue are converted to AMP and further phosphorylated to ATP. Adenosine is directly phosphorylated to AMP, while the breakdown products xanthine and inosine are first ribosylated by PRPP and then converted to AMP. AMP is further phosphorylated via high energy bonds to form adenosine diphosphate (ADP) and ATP.

ATP is necessary for all bodily functions, such as muscle contraction, brain function, digestion and others. A lack of ATP can result in feelings of fatigue, lowered mental capacity, lack of “get up and go” and a lessened quality of life. Barring illness or disease, most persons who are adequately nourished experience fatigue only during extended or extreme exercise.

Fatigued subjects without known cardiovascular, pulmonary or metabolic disorders would be assumed to have adequate ATP levels for normal function. “Baby Boomers” are defined as those persons born between 1946-1964 and are now approximately 80 million in number. Approximately 20% of Americans in this population complain of fatigue, which can interfere with their daily, normal life style, especially when many have achieved success in their profession, with the increased demands that success requires. The perception of fatigue is vague, encompassing symptoms such as tiredness, drowsiness, lethargy, malaise, weakness or a lack of energy. Many baby boomers try to regain a more energetic state in order to continue their careers at a high level and to make their future true “golden years” with a high quality of life. There is no theoretical basis for assuming that older, healthy but sedentary persons such as baby boomers, now aging, would benefit from dietary supplementation.

### SUMMARY OF THE INVENTION

Fatigued, aging subjects without known cardiovascular, pulmonary or metabolic disorders or known increased energy expenditure due to exercise or physical labor, were administered 1.5 or 3.0 grams of D-ribose orally twice a day (bid) for two weeks. Those subjects at the higher dose of six grams of D-ribose per day showed significant improvement in cardiovascular parameters; that is, had improved levels of fitness as assessed by a decrease in cardiac work on moderate exercise, improved aerobic capacity, breathing efficiency and O₂ uptake efficiency. Their self perceived levels of fatigue decreased by an average of 50%.

Subjects at the 1.5 gram dose bid or 3.0 grams of D-ribose a day showed less improvement at two weeks, but when administration was continued for an additional two weeks, positive trends were found in both objective and subjective assessments.

D-ribose, a white powder, was administered in a small amount of water, but can be incorporated in a lozenge, tablet or time release tablet or sprinkled on food. In addition to being administered as a single product, D-ribose may also be administered in combination with other dietary supplements, pharmaceuticals, foods or drinks.

Since the levels of improvements in the parameters measured increased from week one to week two and to week four in the lower dose subjects, it is indicated that improvement would increase and the D-ribose supplement should be administered chronically or long term. Both the number and amount of the dose and the total amount of D-ribose to be ingested each day are important. Each dose may be from 0.100 gram to 3.0 gram repeated at least twice a day. If lower doses are given, the daily total of D-ribose ingested should be from 1.0 to 6.0 grams.

### DESCRIPTION OF THE DRAWINGS

FIG. 1 shows a typical example of the detection of anaerobic threshold.

FIG. 2 shows the anaerobic threshold shift after two weeks of oral D-ribose.

FIG. 3 shows the heart rate to METS ratio at the anaerobic threshold.

FIG. 4 shows the net energy expenditure at the anaerobic threshold.

FIG. 5 graphically displays a summary of SF-36 questionnaire.

FIG. 6 displays a summary of the fatigue questionnaire.

FIG. 7 shows a trend in reducing fatigue.

### DETAILED DESCRIPTION OF THE INVENTION

Many individuals, as they age, slow down, exercise less, eat the same amount of food and gain weight. This cycle feeds on itself and can result in health problems such as heart disease and diabetes. At the end of the work day, which is for many persons a sedentary work, few actively pursue exercise on a regular basis with many complaining of fatigue and tiredness with limited energy and little desire or motivation for exercise. Theoretically, these individuals are probably de-conditioned with undesirable basic metabolism index (BMI) values and “down-regulated” pathways for energy production, feeding into the cycle and perpetuating their inactivity. These negative effects of aging in baby boomers over 45 years of age, occur at a time when the subject has achieved professional success and would like to find a natural means, without side effects, for increasing energy and quality of life. The following study was designed to test whether supplementation with D-ribose can aid in breaking the sedentary cycle so as to improve the fatigue state and even to encourage more physical activity with all its concomitant benefits.

The pilot study focused on older healthy adult aged over 45 years to 65 years. Although the subjects enrolled were 65 or less, the supplementation is recommended for any older adult over 45 up to and including advanced old age.

### Example 1

### Selection of Subjects and Assessment

The pilot study was performed enrolling 20 aging subjects, greater than 45 years of age, who were self-perceived as fatigued and tired as their customary daily state for at least one month, with no strenuous exercise or physical labor to account for the fatigue. No subjects had documented histories of heart/lung or metabolism/endocrine disease, as set out more fully below in the inclusion criteria. The causes of fatigue in aging subjects is unknown. It can be hypothesized that the causes are mental, since lowered cognition and feeling of well being is also common in aging persons. The aforementioned studies of unhealthy persons or persons exercising strenuously found a dose of five to eight grams of D-ribose, taken two to four times per day was recommended to raise or maintain ATP levels. Lower amounts such as in this study, were not found adequate for those subjects. For the healthy, but sedentary and aging subjects for this study, it was expected that they were already at an optimum ATP level. Two doses of oral D-ribose at 1.5 grams or 3.0 grams bid were selected to test the hypothesis that raising ATP levels with D-ribose could have some benefit in improving fatigue. Each subject consumed the oral D-ribose for two weeks, dissolved in a small amount of water. Study assessments were done at baseline, and at one and two weeks during the trial. The lower dose study was continued an additional two weeks. The subjective and objective assessment parameters included: sub-maximal exercise performance, resting and sub-maximal energy expenditure, the SF-36 Quality of Life Assessment and a subjective questionnaire for evaluating fatigue.

### A. Inclusion Criteria.

Subjects of either gender, between the ages of 45 and 65 years of age, who have had no previous clinical diagnosis of pulmonary, cardiac or metabolic disorders, were eligible to be included in the study. The subjects must have been capable of performing a sub-maximal incremental treadmill exercise using cardiopulmonary analysis methods. Mild, untreated per-hypertension (>120/70 but <140/90) was acceptable. Subjects agreed to be compliant with the dose regimen, repeat clinical visits and completion of the study questionnaires. Subjects should not have been taking other adenine nucleotide enhancing supplements such as creatine, carnitine or the like for at least a month before entering the study and during the period of the study. Non-compliance in previous studies or pregnancy were further exclusion criteria.

### B. Assessment.

Subjects were monitored at baseline and during the two week treatment period for their perceived fatigue activity levels. Subjects were asked to rate on a ten point scale (1=near dead to 10=excellent) the following questions: How is your energy? (1=no energy, 10=excellent); How do you sleep? (1=no sleep, 10=8 hours without waking); How is your mental clarity? (1=“brain fog”, 10=good clarity); How bad is your pain?; How is your overall sense of well being? At weeks one and two, subjects were also asked to describe their overall rating of symptoms of fatigue as compared to their symptoms at baseline. The five point scale was; much better, somewhat better; no change; somewhat worse and much worse. The investigators selected endpoints of some assessments to determine whether the subjects remained the same or improved at one and two weeks. The results were represented in a Visual Analog Scale (VAS) for fatigue.

The SF-36 Quality of Life Questionnaire was also used. Subjects were asked to fill out a questionnaire on the normal activities that they participated in. These activities included household chores, walking, yard work and whether the subject routinely climbed stairs. Additionally, subjects were asked how many days in the past week they felt good; missed work or routine chores because of fatigue; how tired they felt and their state on awakening in the morning.

### C. Cardiopulmonary Exercise Testing.

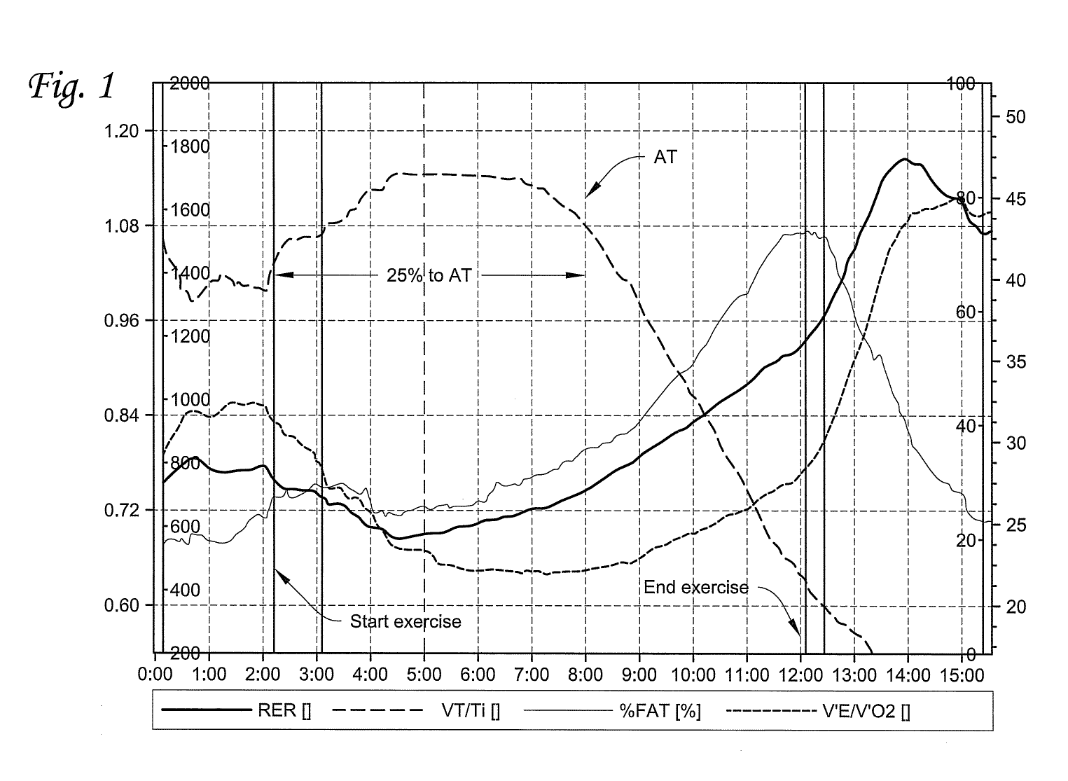

Energy expenditure was calculated both at rest (BMR) and also at the anaerobic threshold (AT) using standard formulae incorporated into CPX-based software. Net energy expenditure was determined by subtracting resting values from those calculated at the subject's AT. In addition, the completed activity log was used to determine potential changes in cumulative (daily and weekly) energy expenditure throughout the first and second weeks while on D-ribose. Further, work efficiency was determined by calculating the reciprocal of aerobic power or the VO₂ to WR ratio, as computed at the anaerobic threshold. FIG. 1 shows an example of the exercise program and the AT point.

The formula for calculation of energy expenditure at the anaerobic threshold was based, in part, on the actual measured resting energy expenditure (RER) and VO₂ at that level of exercise, knowing that a subject can sustain a steady state at the initial phase of the AT, which represents a particular phase of exercise whereby energy metabolism due to an increase in oxygen consumption resulting in a reduction in tissue oxygen perfusion shifts to an anaerobic instead of an oxidative phosphorylation. The AT interval varies from person to person depending on physical condition or training. Individuals who are not trained and relatively deconditioned have a low AT, as compared to elite endurance athletes having a high AT. At the AT, fuel mix for skeletal muscle metabolism is somewhat balanced. This point occurs in the range between 40% to 60% of the maximum VO₂ attained. For example, assuming that equal amounts of fats and carbohydrates are oxidized at an RER of 0.85 just prior to AT onset, energy expenditure can be calculated using the formula VO₂(L/min)×4.862 kcal/min for each liter of oxygen consumed. Likewise, if an individual was at an RER of 0.89 under steady state conditions, their absolute VO₂ in L/min would be multiplied by a factor of 4.911. Net energy expenditure would be calculated subtracting the subject's resting energy expenditure (REE) or BMR. METS or net metabolic equivalents was also used to express the subject's activity level at their AT.

### D. Sub-Maximal Treadmill Exercise Protocol.

For this study, a ramping incremental treadmill exercise protocol was followed. Treadmill speed was incrementally increased by 0.3 mph every minute and grade was increased by 2% each minute, until the patient scores his or her level of exertion to be greater than 14 on the Borg 6-20 scale. The treadmill exercise was increased from 0 mph to 3.0 mph and the elevation increased from 0 to 12%, over the test time of six minutes. The Borg perceived exertion index scale goes from 7 (very, very light) to 13 (somewhat hard) to 19 (very, very hard). No patient was asked to exercise past 14 on the Borg scale. The exercise was stopped at that point and time to reach a Borg scale of 14 was noted.

A more detailed explanation of the various parameters assessed in this application is available in U.S. patent application Ser. No. 11/118,613, the teachings of which are hereby incorporated by reference.

### Example 2

### Pilot Study

A study was performed to test the proposed assessment protocol. Twenty subjects were given 1.5 grams or 3.0 grams of D-ribose bid orally for two weeks. The following results showed the increase or decrease in the parameters measured at the end of the two weeks in those subjects receiving 3.0 grams of D-ribose bid or six grams total per day.

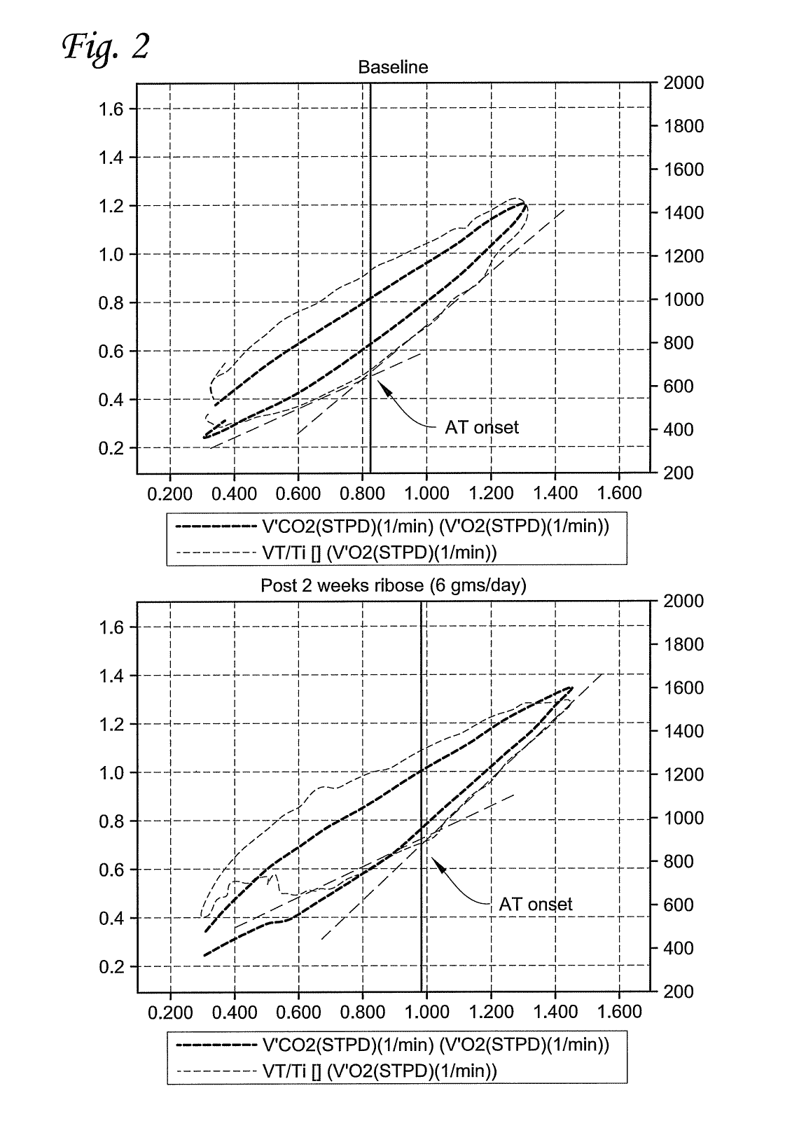

1) Net energy expenditure at the AT onset rose by 32%, with p<0.0005.
2) Resting energy expenditure at the AT onset increased by 8.2%.
3) VO₂ at the AT onset increased by 18%, with p<0.001.
4) Heart rate at the AT onset increased by 9.2%, with p=0.012.
FIG. 2 shows the shift in AT onset after two weeks of D-ribose supplementation and the improvement in parameters. Table 1 summarizes the changes in parameters.

TABLE I
Sub Maximal CPX Testing

| | Visit | Mean change | P value |
|---|---|---|---|
| VO₂ at AT | Week 1 | 1.53 +/− 0.90 | 0.0005 |
| | Week 2 | 2.13 +/− 0.78 | <0.0001 |
| VE Slope | Week 1 | −2.26 +/− 1.69 | 0.0022 |
| | Week 2 | −2.44 +/− 2.24 | 0.0074 |
| O₂ Uptake Slope | Week 1 | 0.17 +/− 0.19 | 0.0215 |
| | Week 2 | 0.24 +/− 0.15 | 0.0008 |
| HR to METS ratio | Week 1 | −3.00 +/− 2.83 | 0.0085 |
| at AT | Week 2 | −3.67 +/− 3.27 | 0.0063 |
| Net Energy | Week 1 | 9.32 +/− 7.67 | 0.0040 |
| Expenditure at AT | Week 2 | 16.23 +/− 6.13 | <0.0001 |

These results indicate that energy efficiency was improved, even over this short term. The average calorie burned from fat substrate at the AT did not change significantly in most subjects, although five subjects showed an actual increase in fat burn calories.

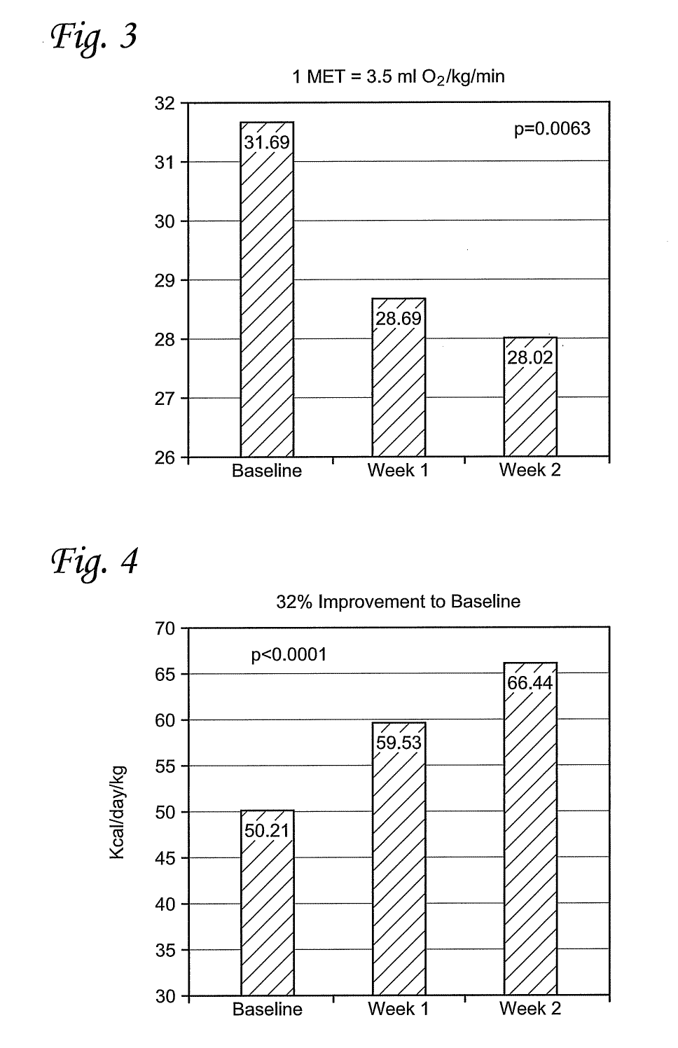

The heart rate to METS ratio decreased by 11.7%, while the ventilatory efficiency slope decreased by 8.5%. The oxygen pulse indexed to inspiratory drive decreased by 8.9%, which possibly indicated less cardiac stroke work. The change in oxygen pulse times the change in expired CO₂ at AT increased by 60.8%, which may be a significant measure of improved efficiency. FIG. 3 is a graphic display of these results, showing the lowered heart rate to METS ratio at AT, indicating that the heart does not have to work as hard at AT to perform as much work. This measure of energy utilization at the cellular level is reflective of an improvement in level of fitness. FIG. 4 again shows net energy expenditure at AT, which is a measure of work performed. Thus, the body is more efficient at energy utilization following two weeks of D-ribose supplementation.

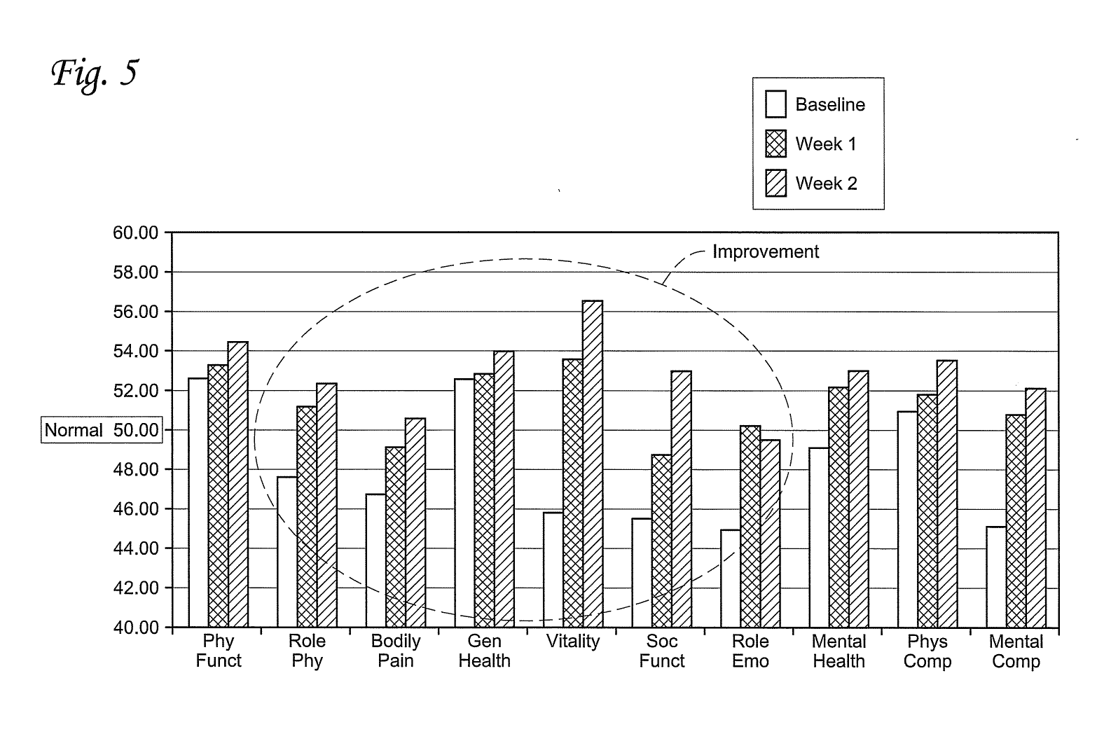

FIG. 5 shows the analyzed results of the SF-36 questionnaire. The baseline questionnaires indicated a frequent occurrence of reduced quality of life. The most significant improvement in symptoms was in “vitality,” while the increases in social functionality, emotional wellbeing, mental health and mental competence were unexpected and had not been seen in previous studies with subjects having cardiovascular disease or healthy subjects exercising past moderate exercise.

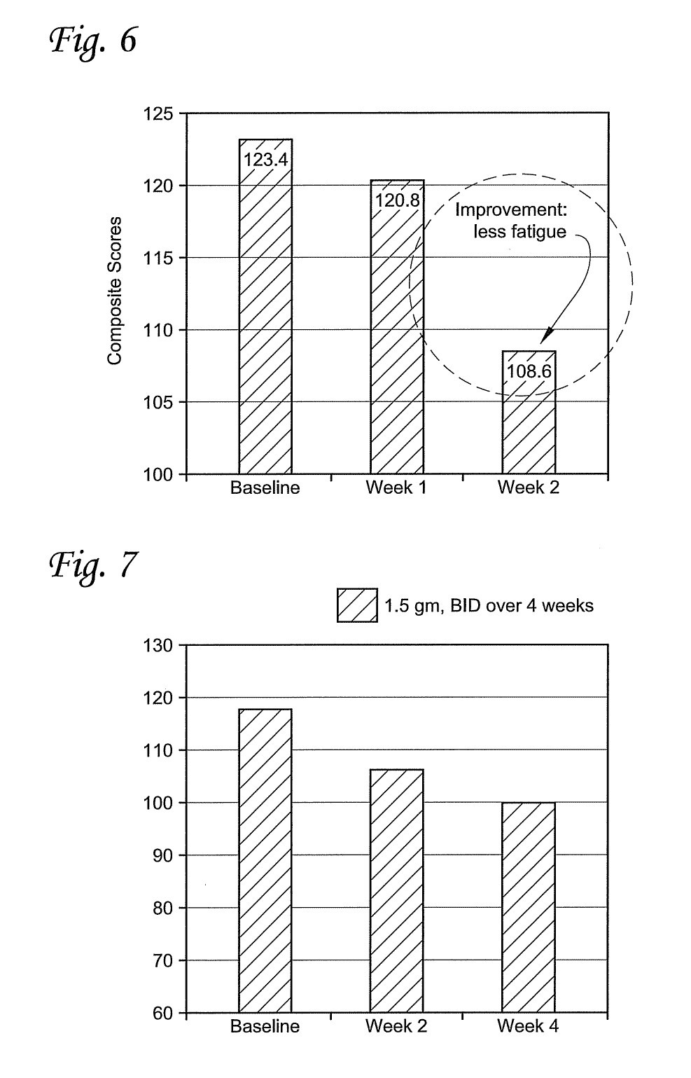

Nine subjects completed the VAS forms of subjective estimate of tiredness. On a scale of 0 (no fatigue) to 10 (very tired), the average score was 47 at baseline and 20 at two weeks. Several observations were of interest: one subject reported an improvement from 80 to 20; another remained at an estimate 50; while those with low initial estimates did not change. FIG. 6 summarizes those results with the composite scores of all participants displayed in a bar graph.

Subjects receiving the lower dose of D-ribose showed positive trends in several parameters. The fatigue questionnaire at two weeks showed a slight reduction in fatigue, although not as significant as that for the higher dose of D-ribose. Therefore, D-ribose administration was continued for an additional two weeks. Continued improvement was found, as shown in FIG. 7. The response to the SF-36 questionnaire showed improvement in symptoms of general health, vitality and mental outlook at four weeks. The objective measures showed less compelling results; there was definitely a positive trend in CPX parameters that increased from two weeks to four weeks. Based on these results it is expected that even lower doses, as low as 0.100 grams, can relieve the symptoms of fatigue in these subjects, provided that the daily total is 1.0 to 6.0 grams of D-ribose. For example, if a subject ingests a dose of 0.100 grams, the subject would take 10 doses a day in order to benefit from the supplementation.

D-ribose ingestion is known to have the potential to cause gastrointestinal distress, including flatulence and diarrhea, and also can lower blood glucose. No subjects in this study, at either the higher or the lower doses, experienced any side effects of D-ribose administration.

In summary, D-ribose administration to aging, healthy but sedentary baby boomers over the age of 45 years, improved subjects vitality and enhanced their quality of life. Surprisingly, subjects reported improvement in mental functions.

We claim:

1. A method comprising the oral administration of an effective amount of D-ribose to aging subjects experiencing fatigue wherein the symptoms of fatigue are relieved.

2. The method of claim 1 wherein the effective amount of D-ribose is 0.1 to 3 grams administered at least twice a day.

3. The method of claim 1 wherein the effective amount of D-ribose is 0.2 to 6 grams per day.

4. The method of claim 1 wherein the symptoms of fatigue comprise tiredness, drowsiness, lethargy, malaise, and/or weakness.

5. The method of claim 1 wherein the effective amount of D-ribose is administered as a single product or in combination with other dietary supplements, pharmaceuticals or food.

6. The method of claim 5 wherein the effective amount of D-ribose is incorporated in a lozenge, tablet or time-release tablet or dissolved in water.

7. The method of claim 1 wherein the effective amount of D-ribose is sprinkled on food.

8. A method comprising the oral administration of an effective amount of D-ribose to aging subjects experiencing a reduced quality of life, wherein the symptoms of a reduced quality of life are relieved.

9. The method of claim 1 wherein the effective amount of D-ribose is 0.1 to 3 grams administered at least twice a day.

10. The method of claim 1 wherein the effective amount of D-ribose is 1 to 6 grams per day.

11. The method of claim 7 wherein the reduced quality of life includes lowered vitality, social functioning, emotional wellbeing, and/or mental competence.
